(19) United States
(12) Patent Application Publication
(10) Pub. No.: US 2019/0060542 A1
(43) Pub. Date: Feb. 28, 2019

(54) Apparatus and Method for Minimally Invasive Implantation of a Heart Assist Device

(71) Applicants: William C. Altman, Houston, TX (US); Boris Leschinsky, Mahwah, NJ (US)

(72) Inventors: William C. Altman, Houston, TX (US); Boris Leschinsky, Mahwah, NJ (US)

(73) Assignee: Corlnnova Incorporated, Houston, TX (US)

(21) Appl. No.: 15/692,345

(22) Filed: Aug. 31, 2017

(51) Int. Cl. A61M 1/12; A61M 1/10

(52) U.S. Cl. CPC A61M 1/122; A61M 1/1008; A61M 1/106

(57) ABSTRACT

The present invention includes a method and device for the minimally invasive implantation in a heart of a deployable device through a left thoracotomy or subxiphoid incision.

### CROSS-REFERENCE TO RELATED APPLICATIONS

Not Applicable.

### TECHNICAL FIELD OF THE INVENTION

The present invention relates generally to methods and devices used in increasing the cardiac output in a patient; and more specifically to methods and devices to aid in minimally invasive implantation of cardiac assist devices.

### STATEMENT OF FEDERALLY FUNDED RESEARCH

Not Applicable.

### INCORPORATION-BY-REFERENCE OF MATERIALS FILED ON COMPACT DISC

Not Applicable.

### BACKGROUND OF THE INVENTION

Without limiting the scope of the invention, its background is described in connection with minimally invasive implantation of cardiac assist devices in patients in need thereof. The left ventricle is the large, muscular chamber of the heart that pumps blood out to the body. Congestive heart failure (CHF) is a condition in which the heart can't pump enough blood to the body's other organs. For people over age 65, it is the number one cause of death, with nearly 290,000 people dying from this disease each year. There are 800,000 people with end-stage CHF and only 2,200 hearts available for transplant each year, leaving a large number of people suffering from this disease. Of that 800,000, about 48,000 are suitable for bridge to transplant and 90,000 patients are suitable for destination therapy. Bridge to transplant refers to the use of ventricle assist devices to sustain severe heart failure patients until a donor heart becomes available and they can receive a transplant. Destination therapy refers to the use of a left ventricular assist device for long-term therapy. It is common for there to be an initial decline in pumping capacity of the heart following heart failure which results in a variety of compensatory mechanisms. The phenomenon of left ventricular remodeling, followed by a change in the wall stress is considered the single most important cause for the worsening of these heart attack patients. Subsequently these patients are rendered helpless and immobile with no options for treatment other than maintenance therapies or placement on a cardiac transplant waiting list.

One treatment for patients who suffer from either a myocardial infarction or CHF is the implantation of direct cardiac compression device (DCCD). Currently, a sternotomy is the preferred method of implantation of the current DCCD, such as the Anstadt cup. This procedure is very painful and results in long recovery times with a high risk of infection. Further, there is a high risk of complications due to the lengthy surgery required for these unstable patients.

U.S. Pat. No. 8,545,387, entitled, “Apparatus and method for minimally invasive implantation of heart assist device,” discloses a method and related apparatus for the minimally invasive implantation about a heart of at least a deployable device. The method comprises the steps of performing a left thoracotomy or subxiphoid incision; obtaining access to the pericardial sac; making a generally linear incision in the pericardial sac; positioning an assembly having an insertion aperture member with an upper ring and a lower ring or flange and insertion tube having therein a deployable device.

U.S. Patent Application Publication No. 20160346449, entitled, “Biomimetic actuation device and system, and methods for controlling a biomimetic actuation device and system,” discloses a biomimetic actuation device includes a flexible substrate, conformable for disposition about an object, defining an apex and a base, bearing at least one soft actuator configured to change state from a first state to a second state upon introduction of a pressurized fluid to an internal volume of the at least one soft actuator. The content of which is incorporated herein by reference.

U.S. Patent Application Publication No. 20160017899, entitled, “Soft actuators and soft actuating devices,” discloses a soft buckling linear actuator is described, including: a plurality of substantially parallel bucklable, elastic structural components each having its longest dimension along a first axis; and a plurality of secondary structural components each disposed between and bridging two adjacent bucklable, elastic structural components; wherein every two adjacent bucklable, elastic structural components and the secondary structural components in-between define a layer comprising a plurality of cells each capable of being connected with a fluid inflation or deflation source; the secondary structural components from two adjacent layers are not aligned along a second axis perpendicular to the first axis; and the secondary structural components are configured not to buckle, the bucklable, elastic structural components are configured to buckle along the second axis to generate a linear force, upon the inflation or deflation of the cells. Methods of actuation using the same are also described. The content of which is incorporated herein by reference.

### SUMMARY OF THE INVENTION

The present invention discloses a method for the minimally invasive implantation in a heart of a deployable device comprising the steps of: performing one of a left thoracotomy or subxiphoid incision; obtaining access to a pericardial sac; providing an the insertion device comprising an insertion tube having an upper aperture at a first end opposite a lower aperture at a second end; making an incision in the pericardial sac dimensioned to receive the upper aperture; providing a deployable device in the insertion device, wherein the deployable device comprising an inner surface opposite an outer surface and an insertion frame removably connected to the deployable device and extends from the lower aperture of the insertion device; pushing the insertion frame from the lower aperture through the upper aperture to move the deployable device through the insertion device; extending a deployable device through the upper aperture to extend inside the pericardial sac; positioning the deployable device about the heart; disengaging the insertion frame from the deployable device; egressing the insertion frame from the insertion device; and removing the insertion device. The deployable device may be preloaded inside the insertion tube. The deployable device may be a biomimetic actuation device comprising: a flexible substrate, defining an apex and a base, bearing at least one soft actuator configured to change state from a first state to a second state upon introduction of a pressurized fluid to an internal volume of the at least one soft actuator, wherein the at least one soft actuator comprises at least one soft actuator disposed curvilinearly along the substrate from the apex of the substrate toward the base of the substrate, at least one soft actuator disposed laterally or circumferentially along the substrate, or a combination of the at least one soft actuator disposed curvilinearly along the substrate and the at least one soft actuator disposed laterally or circumferentially along the substrate, wherein the substrate is conformable for disposition about an object, wherein the at least one soft actuator disposed curvilinearly along the substrate is arranged to deliver torsional forces to an underlying object about which the substrate is disposed, and wherein the at least one soft actuator disposed laterally or circumferentially along the substrate is arranged about the object to deliver compressive forces or extensive forces to an underlying object about which the substrate is disposed. The insertion frame may be connected to the deployable device by a pocket, a fastener, a magnetic connection, a one directional hook, a slot, a tab, or other removable attachment means.

The present invention provides a method for the minimally invasive implantation of a multidirectional compression device around a heart comprising the steps of: obtaining access to a pericardial sac; providing an the insertion device comprising an insertion tube having an upper aperture at a first end opposite a lower aperture at a second end; making an incision in the pericardial sac dimensioned to receive the upper aperture; providing a multidirectional compression device in the insertion device, wherein the multidirectional compression device comprises: a substrate comprising an inner surface adapted to contact the heart opposite an outer surface, one or more first soft actuators oriented in a first direction to compress the heart in a first direction, and one or more second soft actuators oriented in a second direction to compress the heart in a second direction, wherein the one or more first soft actuators, the one or more second soft actuators or both are in operable contact with the substrate, the outer surface or both; an insertion means having an first end in disengageable contact with the multidirectional compression device and a second end that extends from the lower aperture of the insertion device to push the multidirectional compression device through the insertion device; pushing the insertion means into the lower aperture; moving the multidirectional compression device through the upper aperture to move the multidirectional compression device through the insertion device; extending a multidirectional compression device through the upper aperture to extend inside the pericardial sac; positioning the multidirectional compression device about the heart; disengaging the insertion means from the multidirectional compression device; egressing the insertion means from the insertion device; and removing the insertion device.

The one or more first soft actuators may be disposed curvilinearly along the substrate from an apex of the substrate toward a base of the substrate; and the one or more second soft actuators may be disposed laterally or circumferentially along the substrate, wherein the one or more first soft actuators are arranged to deliver torsional forces to the heart, and wherein the one or more second soft actuators are arranged about the heart to deliver compressive forces or extensive forces to the heart. The first direction may be generally vertical or generally horizontal and the second direction may be generally horizontal or generally vertical. The first direction may be between −45 and +45 from vertical and the second direction may be between −45 and +45 from horizontal. The insertion means may be removably connected to the multidirectional compression device by a pocket, a fastener, a magnetic connection, a one directional hook, a slot, a tab, or other removable attachment means.

The present invention provides a method for the minimally invasive implantation of a multidirectional compression device around a heart comprising the steps of: obtaining access to a pericardial sac; providing an the insertion device comprising an insertion tube having an upper aperture at a first end opposite a lower aperture at a second end; making an incision in the pericardial sac dimensioned to receive the upper aperture; providing a multidirectional compression device in the insertion device, wherein the multidirectional compression device comprises: a substrate comprising an inner surface adapted to contact the heart opposite an outer surface, one or more first soft actuators oriented in a first direction to compress the heart in a first direction, and one or more second soft actuators oriented in a second direction to compress the heart in a second direction, wherein the one or more first soft actuators, the one or more second soft actuators or both are in operable contact with the substrate, the outer surface or both; an insertion means having an first end connected to the multidirectional compression device and a second end that extends from the lower aperture of the insertion device to push the multidirectional compression device through the insertion device; pushing the insertion means into the lower aperture; moving the multidirectional compression device through the upper aperture to move the multidirectional compression device through the insertion device; extending a multidirectional compression device through the upper aperture to extend inside the pericardial sac; positioning the multidirectional compression device about the heart; and removing the insertion device.

The one or more first soft actuators may be disposed curvilinearly along the substrate from an apex of the substrate toward a base of the substrate; and the one or more second soft actuators may be disposed laterally or circumferentially along the substrate, wherein the one or more first soft actuators may be arranged to deliver torsional forces to the heart, and wherein the one or more second soft actuators may be arranged about the heart to deliver compressive forces or extensive forces to the heart. The first direction may be generally vertical or generally horizontal and the second direction may be generally horizontal or generally vertical. The first direction may be between −45 and +45 from vertical and the second direction may be between −45 and +45 from horizontal. The insertion means may be a frame, a set of guide rods, a plunger, etc.

The present invention provides a method for the minimally invasive implantation of a multidirectional compression device around a heart comprising the steps of: obtaining access to a pericardial sac; providing an the insertion device comprising an insertion tube having an upper aperture at a first end opposite a lower aperture at a second end; making an incision in the pericardial sac dimensioned to receive the upper aperture; providing a multidirectional compression device in the insertion device, wherein the multidirectional compression device comprises: a substrate comprising an inner surface adapted to contact the heart opposite an outer surface, one or more first soft actuators oriented in a first direction to compress the heart in a first direction, and one or more second soft actuators oriented in a second direction to compress the heart in a second direction, wherein the one or more first soft actuators, the one or more second soft actuators or both are in operable contact with the substrate, the outer surface or both; providing a insertion sleeve in contact with the multidirectional compression device to move the multidirectional compression device around the heart, wherein the insertion sleeve comprises a sleeve adapted to fit the multidirectional compression device and move the multidirectional compression device around the heart, and one or more insertion rods connected to the insertion sleeve, wherein the one or more insertion rods can be moved to move the insertion sleeve which positions the multidirectional compression device about the heart; pushing the one or more insertion rods into the lower aperture; moving the one or more insertion rods and the insertion sleeve through the upper aperture to move the multidirectional compression device through the insertion device; extending the one or more insertion rods and the insertion sleeve through the upper aperture to extend the multidirectional compression device inside the pericardial sac; positioning the multidirectional compression device about the heart; disengaging the insertion sleeve from the multidirectional compression device; egressing the one or more insertion rods and the insertion sleeve from the insertion device; and removing the insertion device.

The one or more first soft actuators may be disposed curvilinearly along the substrate from an apex of the substrate toward a base of the substrate; and the one or more second soft actuators may be disposed laterally or circumferentially along the substrate, wherein the one or more first soft actuators are arranged to deliver torsional forces to the heart, and wherein the one or more second soft actuators are arranged about the heart to deliver compressive forces or extensive forces to the heart. The first direction may be generally vertical or generally horizontal and the second direction may be generally horizontal or generally vertical. The first direction may be between −45 and +45 from vertical and the second direction may be between −45 and +45 from horizontal. The multidirectional compression device may be preloaded inside the insertion tube. The multidirectional compression device comprises a biomimetic actuation device comprising: a flexible substrate, defining an apex and a base, bearing at least one soft actuator configured to change state from a first state to a second state upon introduction of a pressurized fluid to an internal volume of the at least one soft actuator, wherein the at least one soft actuator comprises at least one soft actuator disposed curvilinearly along the substrate from the apex of the substrate toward the base of the substrate, at least one soft actuator disposed laterally or circumferentially along the substrate, or a combination of the at least one soft actuator disposed curvilinearly along the substrate and the at least one soft actuator disposed laterally or circumferentially along the substrate, wherein the substrate is conformable for disposition about an object, wherein the at least one soft actuator disposed curvilinearly along the substrate is arranged to deliver torsional forces to an underlying object about which the substrate is disposed, and wherein the at least one soft actuator disposed laterally or circumferentially along the substrate is arranged about the object to deliver compressive forces or extensive forces to an underlying object about which the substrate is disposed.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are images of the insertion device;

FIGS. 2A and 2B are illustrations of a heart with (2A) an intact pericardium and (2B) with an opening cut at the apex of the pericardial sac;

FIGS. 2C and 2D are illustrations of (2C) the initial insertion of the aperture member of the present invention into the pericardial space and (2D) the final placement of the insertion aperture member of the present invention into the pericardial space;

FIGS. 2E and 2F are illustrations of (2E) an insertion tube of the present invention being attached to the insertion aperture member containing a deployable device, and (2F) the insertion tube and insertion device of the present invention being pulled away from the apex of the heart to allow space for the deployable device;

FIGS. 2G and 2H are illustrations of (2G) the initial deployment of the deployable device through the insertion aperture member of the present invention and (2H) the final placement of the deployable device with the insertion tube of the present invention removed and the insertion aperture member of the present invention left in place;

FIG. 3 illustrates a heart assist device having soft actuators positioned from the apex of the device to the top of the device;

FIG. 4 illustrates a heart assist device having soft actuators positioned from the apex of the device to the top of the device and horizontally around the heart assist device; and

FIG. 5 illustrates a heart assist device positioned around the heart and having soft actuators positioned from the apex of the device to the top of the device and horizontally around the heart assist device.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a device for the implantation of a device around the heart in a minimally invasive fashion. The delivery device includes an insertion aperture member that is moved in position into an aperture in the pericardial sac. Within the insertion aperture member is a deployable device such as a heart assist device that can be extended from the insertion aperture member to encompass the heart. The insertion aperture member can then be removed leaving the heart assist device positioned about the heart. Alternatively, the insertion aperture member may be secured in the position and remain inserted. In another embodiment, the insertion aperture member may include a first framework that is extended from the insertion aperture member to position the pericardial sac to allow the insertion of a heart assist device. The first framework may be removed once the heart assist device is in position about the heart.

In another embodiment, the delivery device includes an insertion device that is moved in position into an aperture in the pericardial sac. The insertion device includes an insertion tube having a first end aperture separated from a second end aperture by an insertion tube. A second insertion tube positioned within the first insertion tube. This multi-tube configuration may be constructed using 2 separate and removable insertion tubes or configured such that the first and second insertion tubes are integrated into a single unit. This multi-tube configuration allows a first framework be inserted in first insertion tube be used to position the pericardial sac so that the second insertion tube may insert the heart assist device about the heart. Similarly, this may be accomplished in other means, e.g., a single insertion tube having internal channels between the inner and outer walls of the insertion tube. The internal channels can house a frame that can be used to position the pericardial sac.

Within the insertion aperture member is a deployable device such as a heart assist device that can be extended from the insertion aperture member to encompass the heart. The insertion aperture member can then be removed leaving the heart assist device positioned about the heart. Alternatively, the insertion aperture member may be secured in the position and remain inserted. In another embodiment, the insertion aperture member may include a first framework that is extended from the insertion aperture member to position the pericardial sac to allow the insertion of a heart assist device. The first framework may be removed once the heart assist device is in position about the heart.

The heart assist device may be may be any device that is placed about the heart. The device can be a direct cardiac compression device (DCCD) such as the Anstadt cup, as is described in U.S. Pat. Nos. 5,119,804, 5,713,954, the heart booster as described in U.S. Pat. No. 5,713,954, heart harness, mesh electrode, heart support device as described in U.S. Pat. No. 3,983,863.

In another embodiment, the present invention may be used to deploy a biomimetic actuation device as described in U.S. Patent Application Publication No. 2016/0346449. The biomimetic actuation device includes a flexible substrate, conformable for disposition about a heart, bearing at least one soft actuator configured to change state from a first state to a second state upon introduction of a pressurized fluid to an internal volume of the at least one soft actuator.

The present invention provides a device for the implantation of a biomimetic actuation device around the heart in a minimally invasive fashion. The delivery device includes an insertion aperture member that is moved in position into an aperture in the pericardial sac, within the insertion aperture member is a deployable biomimetic actuation device that can be extended from the insertion aperture member to encompass the heart. The insertion aperture member can then be removed leaving the heart assist device positioned about the heart. Other heart assist devices include the devices seen in U.S. Pat. Nos. 7,445,593, 7,935,045, 8,187,160, 8,944,986, 9,510,746, and U.S. Patent Application Publication Nos. 2011/0021864, 2004/0167375, 2006/0142634, and 2016/0346449 and similar patents and patent applications (each of which is incorporated herein by reference).

A deployable device is implanted in a heart in accordance with the method of the present invention, using the assembly hereinabove described, as follows. A normal left thoracotomy or subxiphoid incision is performed to allow access.

The present invention provides a mechanism to insert the heart assist device around the heart in a minimally invasive manner. This is accomplished by an insertion means that allows the assist device to be inserted into the delivery device and pushed through the insertion device.

The insertion device may include an insertion tube that houses a single wire frame that removably connects to a heart assist device and extends from the external end of the insertion tube located outside the body cavity. In one embodiment, the heart assist device wire frame is a wire frame of 3, 4, 5, 6, 7, 8, 9, 10 or more wires that removably attach to different locations on the heart assist device and extend away from the heart assist device to allow the wires to extend from the external aperture of the insertion device when the heart assist device is loaded into the insertion device. In some embodiments, the wire frame includes one or more spacers that stabilize the wire frame and maintain the location of each wire relative to the other wires. In addition, the spacer provides rigidity to the wire frame to allow the wire frame to be pushed through the insertion device moving the heart assist device through the insertion device and around the heart.

Alternatively, the insertion device may include an insertion tube that houses a multipart wire frame work having a heart assist device wire frame that removably connects to a heart assist device and an insertion frame that connects to the heart assist device wire frame and extends from the external end of the insertion tube located outside the body cavity. The wire frame may be made from different types of wires with different strengths and characteristics. For example, the wire frame that is connected to the heart assist device may be from a lighter weight/strength wire that causes the wire frame and in turn the heart assist device to flare out to accommodate the heart. This may be a memory metal frame or a bent frame that demonstrates a flared configuration at rest. The wire frame may be connected to a hub, spacer or linker that connects the flaring wire frame to an insertion mechanism. The insertion mechanism may include wires having a greater structural rigidity to allow the moving of the heart assist device in the insertion device. This provides the rigidity to move the heart assist device into and through the insertion device and around the heart while maintaining the flexibility to flare out around the heart for deployment. The number, size and flexibility of the insertion wires do not have correlate to the number, size and flexibility of the wires that induce the flaring. Alternatively, the insertion wire frame may be in the form of rods, tubes, or other structures that accommodates the pushing through the insertion tube.

Although the terminology used herein denotes “wire” this is a general term and includes metal materials, alloy materials, polymer materials, synthetic materials, carbon fiber materials, nanoparticle materials, nanofiber materials, hybrid components of metals and polymers and any other material that can accomplish these uses. For example, the wire may be a polymer filament, woven carbon fiber strands, a polymer or carbon fiber tube, etc.

Any of the embodiments may have a connection device, connection ring, adaptor ring or hub, stabilizer ring or hub, wire connectors, or any other device that is positioned to hold the individual members of the heart assist device wire frame and/or the insertion frame in a specific position relative to each other. For example, the heart assist device wire frame may have wire interconnectors between adjacent members to afford rigidity and reinforce flaring of the device; a polymer hub may allow the wires to pass through the polymer hub at a position about the apex of the heart assist device to stabilize the heart assist device wire frame; an adaptor hub may be positioned to connect the frame of the heart assist device wire frame to the frame of the insertion frame; a stabilizer hub may allow the insertion frame wires to pass through the stabilizer hub to stabilize the insertion frame for pushing through the insertion tube; and embodiments may have any combination of these features.

The heart assist device wire frame may be connected to the heart assist device such that it is not removable and extends from the body cavity. In such cases, the heart assist device wire frame may be an integral part of the heart assist device and may not be detachable.

The heart assist device may be in communication with the heart assist device wire frame in a removable manner. This may be accomplished through various mechanisms. For example, the heart assist device wire frame may be adapted to fit into packets in the heart assist device to allow the device to be pushed through the insertion tube and around the heart. Once in position the heart assist device wire frame may be pulled away from the heart assist device disengaging the heart assist device wire frame from the heart assist device. The heart assist device wire frame may then be withdrawn from the body.

In another embodiment, the heart assist device wire frame may include removable connection means, e.g., removable fasteners that can be selectively engaged, magnetic fasteners, one way fasteners (similar to a J hook) that hold the heart assist device when pushing toward the heart but does not engage the heart assist device when being pulled away from the heart to allow for removal of the heart assist device wire frame.

In another embodiment, the heart assist device wire frame may be partially removable. The heart assist device wire frame may be disassociated from the heart assist device once it is positioned around the heart.

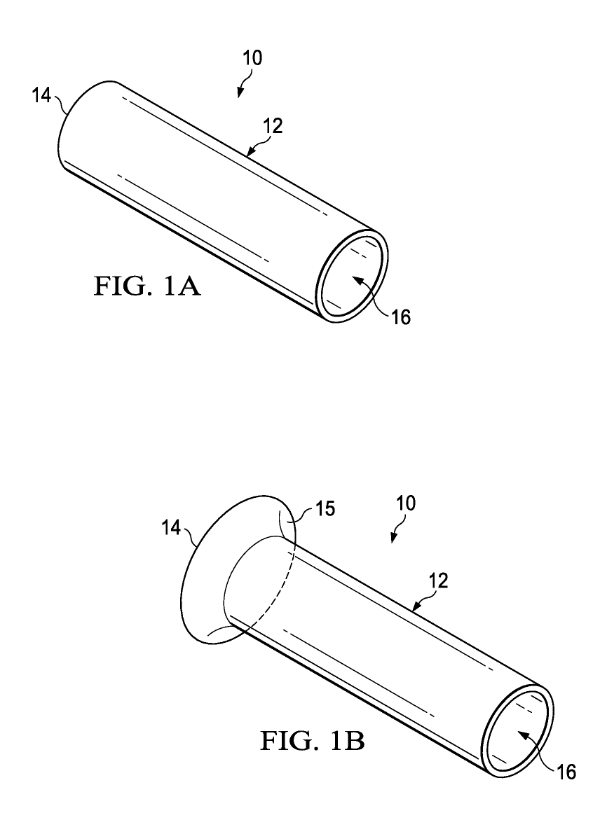

FIGS. 1A and 1B are images of the insertion device 10 of the instant invention. FIG. 1A is an insertion tube 12 having a first aperture 14 having the diameter consistent with the diameter of the insertion tube 12. Insertion tube opening 16 allows access to the interior of the interior of the insertion tube 12 and the length of the insertion tube 12 may be any length necessary. FIG. 1B is an insertion tube 12 having a first aperture 14 having the diameter that is larger than the diameter of the insertion tube 12 to create an aperture flair 15. Insertion tube opening 16 allows access to the interior of the interior of the insertion tube 12 and the length of the insertion tube 12 may be any length necessary.

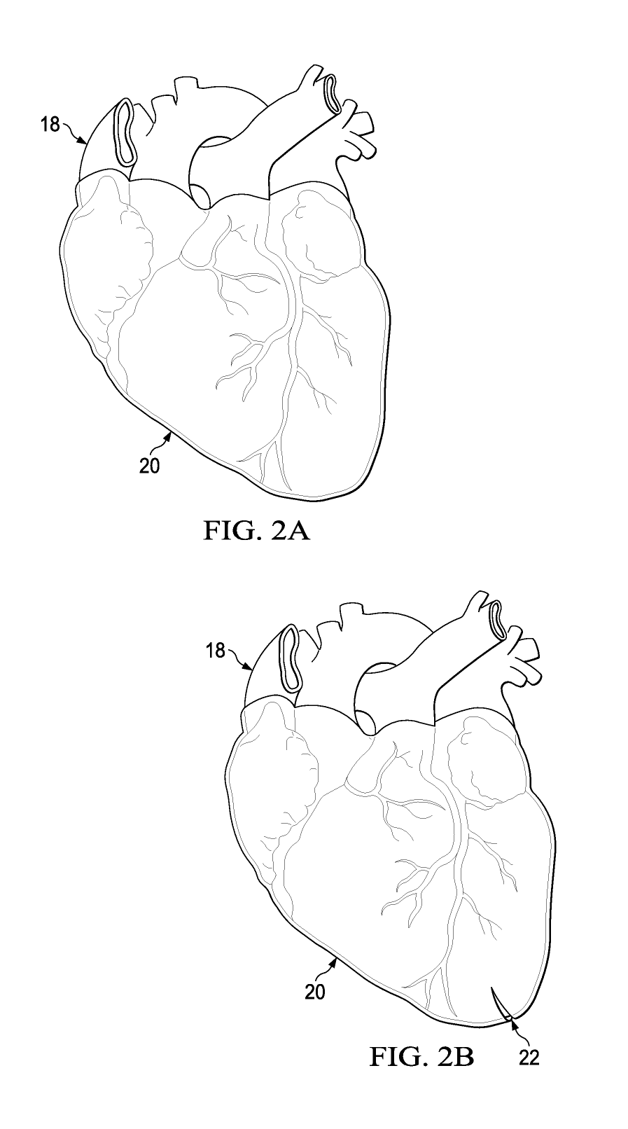

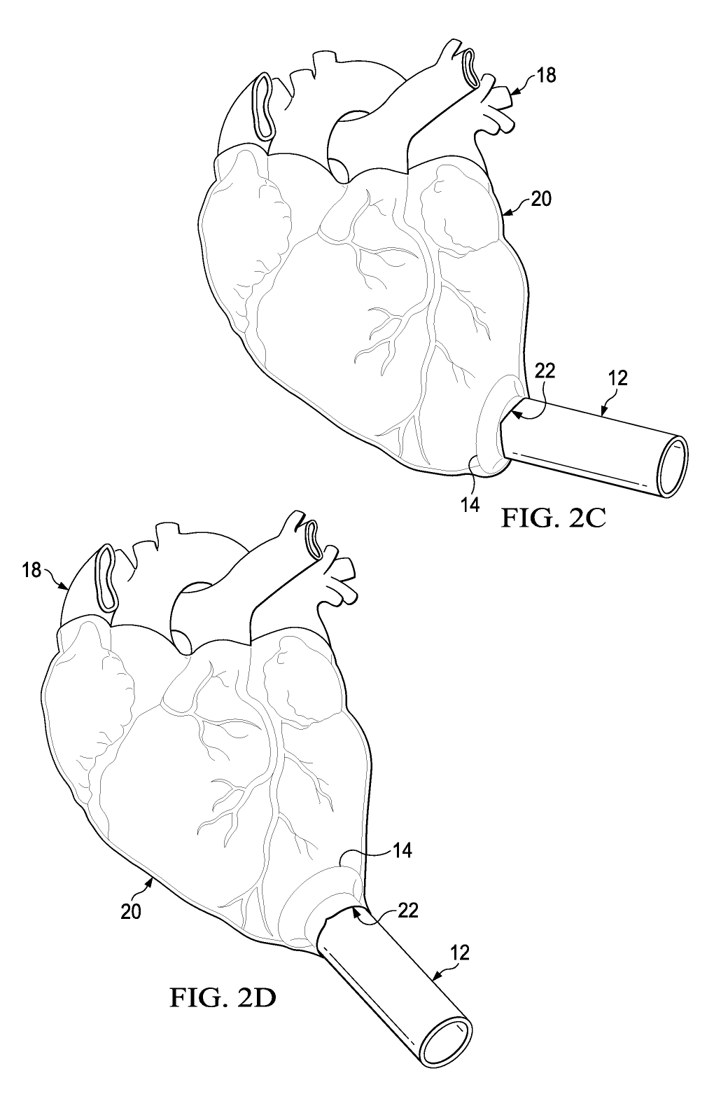

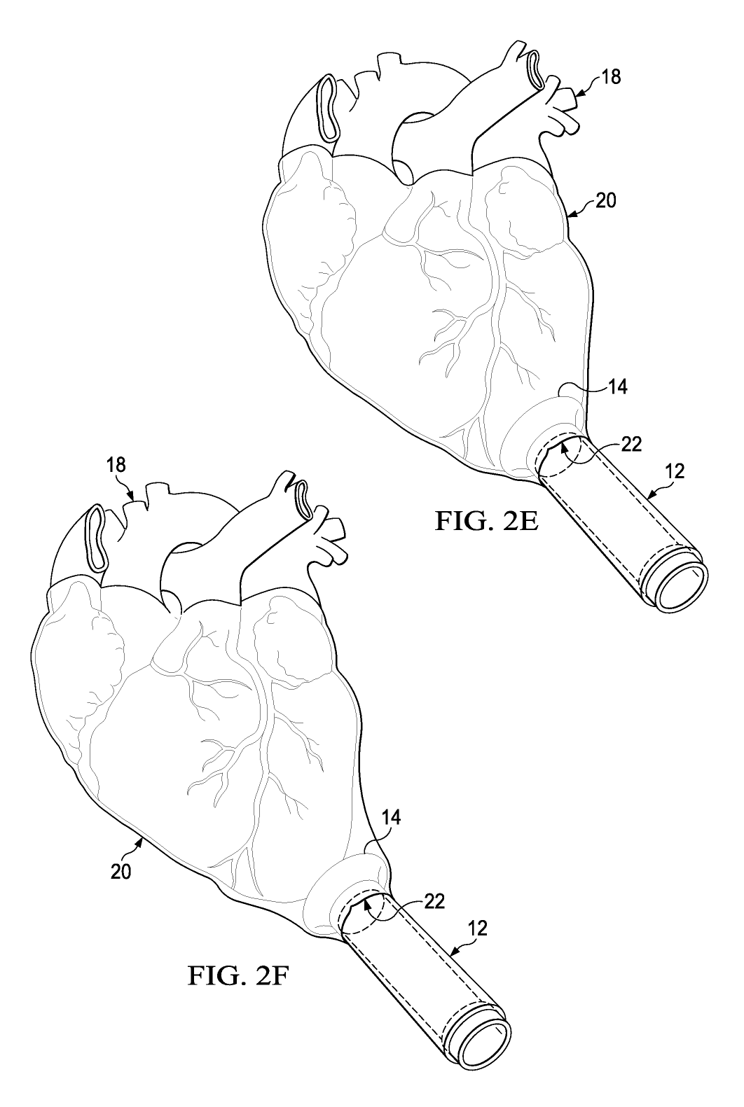

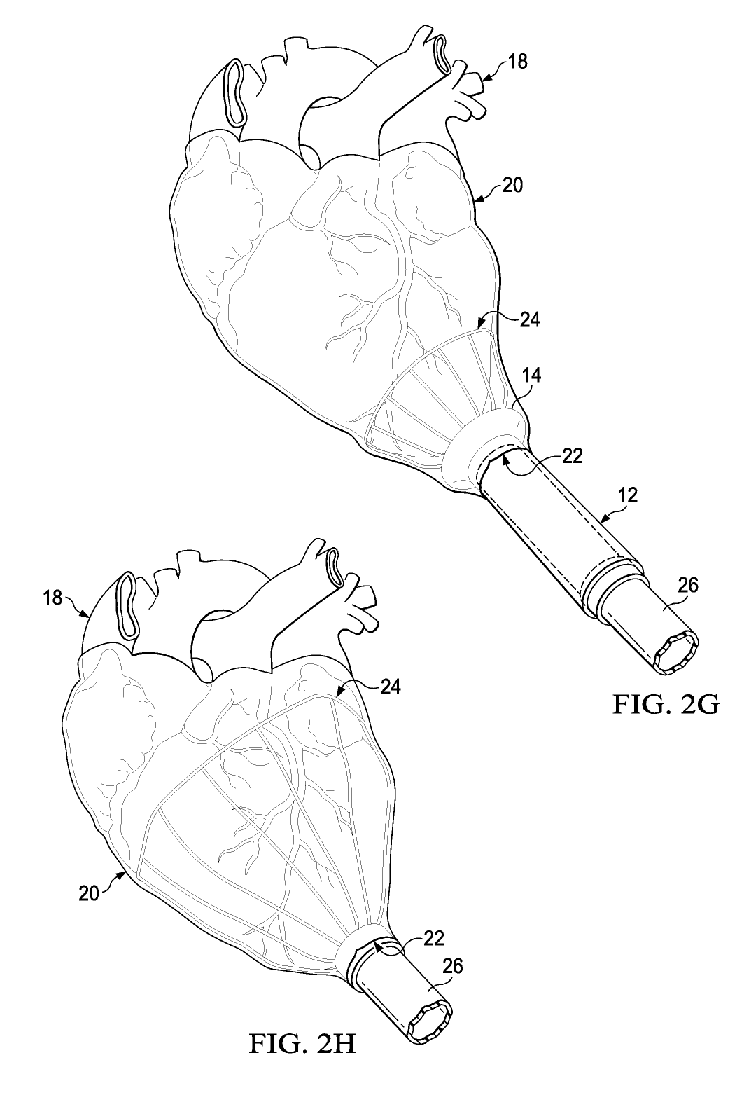

FIGS. 2A and 2B are illustrations of a heart 18 with (FIG. 2A) an intact pericardium and (FIG. 2B) with an opening cut at the apex of the pericardial sac 20. Once access to the pericardial sac 20 has been obtained, a small incision 22 is made in the pericardial sac 20 as hereinbefore described and as seen in FIG. 2B. The insertion aperture member (not shown) is folded and grasped by long forceps and then inserted inside the pericardial sac 20.

FIGS. 2C and 2D are illustrations of the initial insertion of the aperture member of the present invention into the pericardial space and the final placement of the insertion device 10 of the present invention into the pericardial space (not shown). The first aperture 14 is pushed through the incision 22 in the pericardium sac 20. The first aperture 14 and a portion of the insertion device 10 may be slid completely through the incision 22 in the pericardium sac 20 along the anterior of the heart wall (not shown). Once the first aperture 14 is completely inside the pericardial space, it preferably is slid back toward the apex of the pericardium sac 20 and the flaps of the opening in the pericardium sac 20 may be pulled around the insertion tube 12. The elasticity of the pericardium assists in maintaining the opening and keeping the insertion tube 12 in place as seen in FIG. 2D. The insertion device 10 contains a deployable device (not shown) such as a heart assist or DCCD, is then inserted through the first aperture 14.

FIGS. 2E and 2F are illustrations of an insertion device 10 of the present invention being inserted into the incision 22 and housing a deployable device (not shown) with the insertion device 10 being pulled away from the apex of the heart 18 to allow space for the deployable device (not shown). Once the insertion device 10 is positioned the entire insertion tube 12 can be pulled away from the heart 18 to create space between the pericardial sac 20 and the apex of the heart as seen in FIG. 2F. If the opening in the pericardial sac 20 is too large, the insertion device 10 and first aperture 14 may accidentally be pulled out of the incision 22. Sutures (not shown) can be used to help hold the insertion device 10 and first aperture 14 in place. Once the pericardial sac 20 is lifted away from the apex of the heart, the deployable device (not shown), such as a heart assist or DCCD may be deployed as seen in FIGS. 2G and 2H.

FIGS. 2G and 2H are illustrations of the initial deployment of the deployable device 24 through the first aperture 14 of the insertion device 10 and the final placement of the deployable device 24 with the insertion tube 12 removed and the deployable device 24 left in place. The control conduit 26 remains attached to the deployable device 24 and operably controls the activity of the deployable device 24 to assist the function of the heart 18. The deployable device 24 (e.g., DCCD), is adapted to surround the heart 18 and, e.g., to compress the ventricle. The present invention further includes a deploying mechanism adapted to deploy and retract the deployable device 24 through the insertion tube 12 and out of the first aperture 14 and around the heart 18.

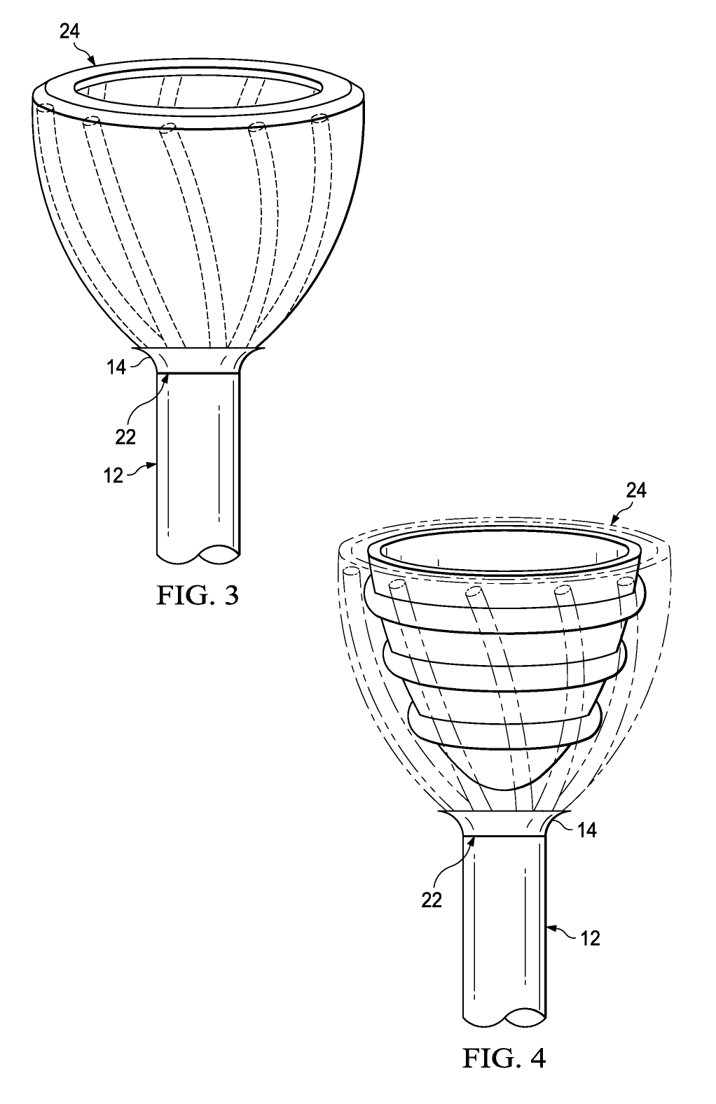

FIG. 3 illustrates a heart assist device having soft actuators positioned from the apex of the device to the top of the device;

FIG. 4 illustrates a heart assist device having soft actuators positioned from the apex of the device to the top of the device and horizontally around the heart assist device; and

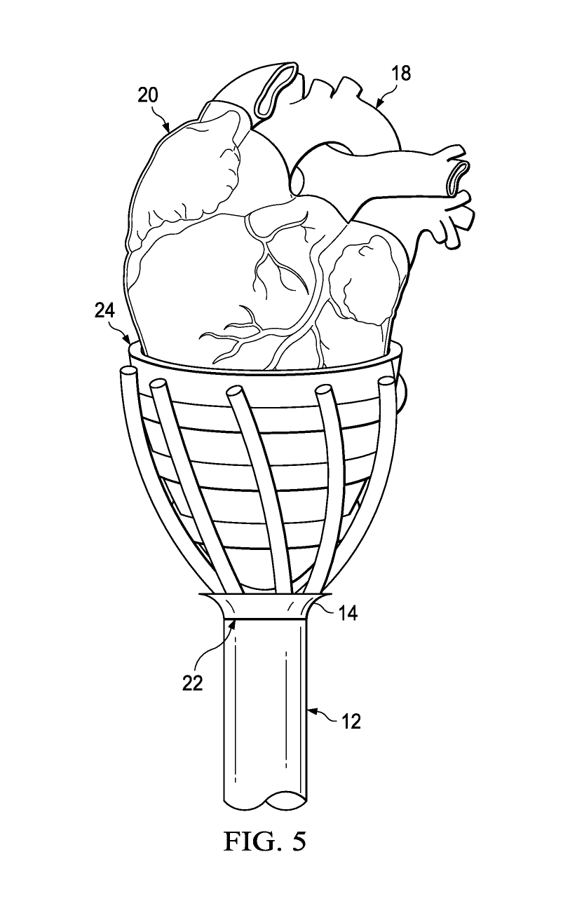

FIG. 5 illustrates a heart assist device positioned around the heart and having soft actuators positioned from the apex of the device to the top of the device and horizontally around the heart assist device.

The present invention provides a method for the minimally invasive implantation of a multidirectional compression device around a heart comprising the steps of: obtaining access to a pericardial sac; providing an insertion device comprising an insertion tube having an upper aperture at a first end opposite a lower aperture at a second end; making an incision in the pericardial sac dimensioned to receive the upper aperture; providing a multidirectional compression device in the insertion device, wherein the multidirectional compression device comprises: a substrate comprising an inner surface adapted to contact the heart opposite an outer surface, one or more first soft actuators oriented in a first direction to compress the heart in a first direction, and one or more second soft actuators oriented in a second direction to compress the heart in a second direction, wherein the one or more first soft actuators, the one or more second soft actuators or both are in operable contact with the substrate, the outer surface or both; an insertion means having a first end in disengageable contact with the multidirectional compression device and a second end that extends from the lower aperture of the insertion device to move the multidirectional compression device through the insertion device; positioning the insertion means in the insertion device; moving the multidirectional compression device through the upper aperture toward the heart; extending the multidirectional compression device through the upper aperture to extend inside the pericardial sac; expanding a distal end of the multidirectional compression device to envelop the heart; positioning the multidirectional compression device about the heart; disengaging the insertion means from the multidirectional compression device; and removing the insertion device. The multidirectional compression device may have various configurations that include supports or actuators that extend from the apex of the device to the top of the device or extend partially from the apex toward the top of the device. In some instances, the device may have a combination of different length supports, different diameter supports, inflatable supports, flexible supports, wire supports, etc. In some embodiments, the supports or actuators are used to add strength in a specific direction to allow the device to be inserted; in other embodiments, a separate device is added or connected to the device to insert the device. The device can use a combination of actuators, wires, and external insertion rods to move the device for insertion. For example, one embodiment includes a multidirectional compression device that has inflatable actuators oriented from the apex to the top of the multidirectional compression device which are inflated to provide rigidity to the multidirectional compression device so that it can be inserted deployed. The multidirectional compression device can be connected to a means for pushing the multidirectional compression device through the insertion tube. This means may be rigid supports that connect to the multidirectional compression device, a framework that fits around the multidirectional compression device and guides the multidirectional compression device into position, a intercalated frame work or extended support that allows the multidirectional compression device to be moved through the insertion device.

As noted herein, there are a number of features and advantages of the apparatus and method of the present invention, including: the apparatus is adapted to permit deployment of a deployable device 24, such as a DCCD or any other device described and incorporated herein, through a small left thoracic incision (not shown) or subxiphoid incision (not shown). The insertion device 10 permits deployment of a deployable device 24 such as a heart assist or DCCD into the pericardial space inside the pericardial sac 20 via a small incision located at the apex of the pericardium; the insertion aperture member of the apparatus of the present invention being collapsible to fit inside the pericardial opening and then expand to a dimension greater than that of the opening; the apparatus being adapted to stabilize the pericardial sac without the need for suturing—however, suturing may be incorporated during the implantation procedure; the apparatus comprising a minimal number of components—including an insertion aperture member which stabilizes the pericardial sac and an insertion tube which houses a deployable device and is adapted to guide the deployable device during deployment; and the insertion aperture member and insertion tube being separated or integrated into a single unit. Once in place, the apparatus of the present invention allows a surgeon to lift the pericardial sac away from the apex of the heart in order to gain space required for deployment of a deployable device—there is no need for suturing to accomplish this task, although suturing may be applied.

If the two components of the apparatus of the present invention are separate components, once the insertion aperture member is in place, it can be attached to the insertion tube containing the deployable device and after the attachment of the insertion tube to the insertion aperture member, the deployable device may be deployed around the heart under the pericardial sac. After deployment of the deployable device, the insertion tube may be removed from the insertion aperture member. Further, the insertion aperture member may be removed or may be left in place to allow for future access to the pericardial space. The apparatus of the present invention is made of biocompatible materials including from the group including, but not limited to silicon, polytetrafluoroethylene (PTFE), ultra high molecular weight polyethylene (UHMWPE), a suitable polymer, elastomer or rubber material and may be coated with therapeutic agents, drugs or substances to assist in healing.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein.

Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the concept, spirit, and scope of the invention as defined by the appended claims.

What is claimed is:

1. A method for the minimally invasive implantation of a multidirectional compression device around a heart comprising the steps of:
obtaining access to a pericardial sac;
providing an insertion device comprising an insertion tube having an upper aperture at a first end opposite a lower aperture at a second end;
making an incision in the pericardial sac dimensioned to receive the upper aperture;
providing a multidirectional compression device in the insertion device, wherein the multidirectional compression device comprises:
a substrate comprising an inner surface adapted to contact the heart opposite an outer surface,
one or more first soft actuators oriented in a first direction to compress the heart in a first direction, and
one or more second soft actuators oriented in a second direction to compress the heart in a second direction, wherein the one or more first soft actuators, the one or more second soft actuators or both are in operable contact with the substrate, the outer surface or both;
an insertion means having a first end in disengageable contact with the multidirectional compression device and a second end that extends from the lower aperture of the insertion device to move the multidirectional compression device through the insertion device;
positioning the insertion means in the insertion device;
moving the multidirectional compression device through the upper aperture toward the heart;
extending the multidirectional compression device through the upper aperture to extend inside the pericardial sac;
expanding a distal end of the multidirectional compression device to envelop the heart;
positioning the multidirectional compression device about the heart;
disengaging the insertion means from the multidirectional compression device; and
removing the insertion device.

2. The method of claim 1, wherein the one or more first soft actuators, one or more second soft actuators or both are pressurizable during insertion to provide at least partial rigidity to the multidirectional compression device during insertion to allow the device to be inserted.

3. The method of claim 1, further comprising a set of insertion guides in contact generally vertically with the multidirectional compression device to provide rigidity to the multidirectional compression device during insertion.

4. The method of claim 3, wherein the set of insertion guides are inflatable passages, inflatable tubes, inflatable rods, metal guides, polymer passages, composite passages, or a combination thereof to provide at least partial rigidity to the multidirectional compression device during insertion.

5. The method of claim 1, wherein the one or more first soft actuators are disposed curvilinearly along the substrate from an apex of the substrate toward a base of the substrate; and the one or more second soft actuators are disposed laterally or circumferentially along the substrate, wherein the one or more first soft actuators are arranged to deliver torsional forces to the heart, and wherein the one or more second soft actuators are arranged about the heart to deliver compressive forces or extensive forces to the heart.

6. The method of claim 1, wherein the first direction is generally vertical or generally horizontal and the second direction is generally horizontal or generally vertical.

7. The method of claim 1, wherein the first direction is between −45 and +45 from vertical and the second direction is between −45 and +45 from horizontal.

8. The method of claim 1, wherein the insertion means is a frame, a set of guide rods, a plunger, a push rod or a combination thereof.

9. The method of claim 1, wherein the insertion means is removably connected to the multidirectional compression device by a pocket, a fastener, a magnetic connection, a one directional hook, a slot, a tab, the set of insertion guides or other removable attachment means.

10. A method for the minimally invasive implantation of a multidirectional compression device around a heart comprising the steps of:
obtaining access to a pericardial sac;
providing an insertion device comprising an insertion tube having an upper aperture at a first end opposite a lower aperture at a second end;
making an incision in the pericardial sac dimensioned to receive the upper aperture;
providing a multidirectional compression device in the insertion device, wherein the multidirectional compression device comprises:
a substrate comprising an inner surface adapted to contact the heart opposite an outer surface,
one or more first soft actuators oriented in a first direction to compress the heart in a first direction, and
one or more second soft actuators oriented in a second direction to compress the heart in a second direction, wherein the one or more first soft actuators, the one or more second soft actuators or both are in operable contact with the substrate, the outer surface or both;
an insertion means having a first end connected to the multidirectional compression device and a second end that extends from the lower aperture of the insertion device to push the multidirectional compression device through the insertion device;
positioning the insertion means in the insertion device;
moving the multidirectional compression device through the upper aperture toward the heart;
at least partially pressurizing the one or more first soft actuators, the one or more second soft actuators or both to provide at least partial rigidity to the multidirectional compression device to allow the multidirectional compression device to be inserted;
extending a multidirectional compression device through the upper aperture to extend inside the pericardial sac;
expanding a distal end of the multidirectional compression device to envelop the heart;
positioning the multidirectional compression device about the heart; and
removing the insertion device.

11. The method of claim 10, wherein the one or more first soft actuators is disposed curvilinearly along the substrate from an apex of the substrate toward a base of the substrate; and the one or more second soft actuators disposed laterally or circumferentially along the substrate, wherein the one or more first soft actuators are arranged to deliver torsional forces to the heart, and wherein the one or more second soft actuators are arranged about the heart to deliver compressive forces or extensive forces to the heart.

12. The method of claim 10, wherein the first direction is generally vertical or generally horizontal and the second direction is generally horizontal or generally vertical.

13. The method of claim 10, wherein the first direction is between −45 and +45 from vertical and the second direction is between −45 and +45 from horizontal.

14. A method for the minimally invasive implantation of a multidirectional compression device around a heart comprising the steps of:
obtaining access to a pericardial sac;
providing an insertion device comprising an insertion tube having an upper aperture at a first end opposite a lower aperture at a second end;
making an incision in the pericardial sac dimensioned to receive the upper aperture;
providing a multidirectional compression device in the insertion device, wherein the multidirectional compression device comprises:
a substrate comprising an inner surface adapted to contact the heart opposite an outer surface,
a set of support guides in contact with the multidirectional compression device to provide rigidity to the multidirectional compression device during insertion,
one or more first soft actuators oriented in a first direction to compress the heart in a first direction, and
one or more second soft actuators oriented in a second direction to compress the heart in a second direction, wherein the one or more first soft actuators, the one or more second soft actuators or both are in operable contact with the substrate, the outer surface or both;
providing a insertion sleeve in contact with the multidirectional compression device to move the multidirectional compression device around the heart, wherein the insertion sleeve comprises a sleeve adapted to fit the multidirectional compression device and move the multidirectional compression device around the heart, and one or more insertion rods connected to the insertion sleeve, wherein the one or more insertion rods can be moved to move the insertion sleeve which positions the multidirectional compression device about the heart;
pushing the one or more insertion rods into the lower aperture;
moving the one or more insertion rods and the insertion sleeve through the upper aperture to move the multidirectional compression device through the insertion device;
at least partially inflating the set of inflatable insertion guides to provide at least partial rigidity to the multidirectional compression device to allow the multidirectional compression device to be inserted;
extending the one or more insertion rods and the insertion sleeve through the upper aperture to extend the multidirectional compression device inside the pericardial sac;
expanding a distal end of the multidirectional compression device to envelop the heart;
positioning the multidirectional compression device about the heart;
disengaging the insertion sleeve from the multidirectional compression device;
egressing the one or more insertion rods and the insertion sleeve from the insertion device; and
removing the insertion device.

15. The method of claim 14, wherein the set of support guides comprise inflatable passages, inflatable tubes, inflatable rods or a combination thereof to provide rigidity to the multidirectional compression device during insertion.

16. The method of claim 14, wherein the set of support guides comprise vertical supports comprising a polymer, a metal, an alloy, a composite, or a combination thereof.

17. The method of claim 14, wherein the one or more first soft actuators is disposed curvilinearly along the substrate from an apex of the substrate toward a base of the substrate; and the one or more second soft actuators disposed laterally or circumferentially along the substrate, wherein the one or more first soft actuators are arranged to deliver torsional forces to the heart, and wherein the one or more second soft actuators are arranged about the heart to deliver compressive forces or extensive forces to the heart.

18. The method of claim 14, wherein the first direction is generally vertical or generally horizontal and the second direction is generally horizontal or generally vertical.

19. The method of claim 14, wherein the first direction is between −45 and +45 from vertical and the second direction is between −45 and +45 from horizontal.

20. The method of claim 14, wherein the multidirectional compression device comprises a biomimetic actuation device comprising: a flexible substrate, defining an apex and a base, bearing at least one soft actuator configured to change state from a first state to a second state upon introduction of a pressurized fluid to an internal volume of the at least one soft actuator, wherein the at least one soft actuator comprises at least one soft actuator disposed curvilinearly along the substrate from the apex of the substrate toward the base of the substrate, at least one soft actuator disposed laterally or circumferentially along the substrate, or a combination of the at least one soft actuator disposed curvilinearly along the substrate and the at least one soft actuator disposed laterally or circumferentially along the substrate, wherein the substrate is conformable for disposition about an object, wherein the at least one soft actuator disposed curvilinearly along the substrate is arranged to deliver torsional forces to an underlying object about which the substrate is disposed, and wherein the at least one soft actuator disposed laterally or circumferentially along the substrate is arranged about the object to deliver compressive forces or extensive forces to an underlying object about which the substrate is disposed.
